(19) United States
(12) Patent Application Publication
(10) Pub. No.: US 2013/0217615 A1
(43) Pub. Date: Aug. 22, 2013

(54) COMBINATION TREATMENT OF MAJOR DEPRESSIVE DISORDER

(71) Applicants: Robert A. Lasser, Florence, KY (US); Timothy M. Whitaker, Florence, KY (US); Jeff Jonas, Florence, KY (US)

(72) Inventors: Robert A. Lasser, Florence, KY (US); Timothy M. Whitaker, Florence, KY (US); Jeff Jonas, Florence, KY (US)

(73) Assignee: SHIRE LLC, Florence, KY (US)

(21) Appl. No.: 13/881,230

(22) PCT Filed: Oct. 28, 2011
(86) PCT No.: PCT/GB11/52103

§ 371 Date: Apr. 24, 2013

Related U.S. Application Data
(60) Provisional application No. 61/407,700, filed on Oct. 28, 2010.

(51) Int. Cl. A61K 31/343; A61K 31/165; A61K 45/06; A61K 38/02

(52) U.S. Cl. CPC A61K 31/343; A61K 38/02; A61K 31/165; A61K 45/06
USPC 514/1.1; 514/469; 514/626

(57) ABSTRACT

A method is provided for treating depression, and particularly major depression,by administering an effective amount of an SSRI medication that is augmented by administering an effective amount of an amphetamine prodrug, such as lisdexamfetamine, to patients receiving an SSRI medication. In particular, the amphetamine prodrug is administered to augment the effects of an SSRI medication in patients who respond poorly to the effects of the SSRI medication.

### CROSS-REFERENCE TO RELATED APPLICATION

This patent application claims the benefit of priority from U.S. Provisional Patent Application No. 61/407,700 filed Oct. 28, 2010, the contents of which are incorporated herein by reference.

### FIELD OF INVENTION

The present invention relates to methods of enhancing the effectiveness of selective serotonin reuptake inhibitors (SSRI's) in the treatment of depression using stimulants. In particular, the invention provides improved methods for treating depressive conditions such as major depressive disorder using combinations of SSRI's and amphetamines or related compounds.

### BACKGROUND OF THE INVENTION

Depression is a serious illness that affects a person's family, work or school life, sleeping and eating habits, and general health. Its impact on functioning and well-being has been equated to that of major chronic medical conditions such as diabetes.

Major Depressive Disorder (MDD), also known as major depression, is a neurobiological disorder characterized by changes in mood, concentration, sleep, appetite, interest, or motivation which last for at least 2 weeks, resulting in functional disability (e.g., less work productivity, increased absenteeism from school or work, reduced social activity, difficulty performing every-day tasks). MDD is considered ‘unipolar’ depression, distinct from ‘bipolar depression’ in which patients longitudinally experience symptoms of both depression and mania. Inadequate response in MDD is recognized as a clinical entity by health authorities, with clear pathways for approval of therapeutic interventions.

Both medication therapy and psychotherapy (‘talk therapy’) are used to alleviate the symptoms of MDD. Research indicates that up to two-thirds of individuals continue to experience symptoms and related disability following initial treatment. These two-thirds are described as maintaining an ‘inadequate response’ in MDD. In research studies, ‘inadequate response’ is defined as experiencing less than 50% improvement in depressive symptoms and lack of improvement to a threshold of overall symptom intensity.

All currently available antidepressants are known to acutely enhance some aspect of monoaminergic function. Most are reuptake inhibitors of one or more of these monoamines.

Selective serotonin reuptake inhibitors (“SSRIs”) are a class of compounds typically used as antidepressants. SSRIs increase the extracellular level of serotonin (“5-HT”) in tissues of the nervous system receiving serotonergic innervation by inhibiting its reuptake into the presynaptic cell, increasing the level of 5-HT available to bind to the postsynaptic receptor. They have varying degrees of selectivity for the other monoamine transporters, with pure SSRIs having only weak affinity for the norepinephrine (“NE”) and dopamine (“DA”) transporter.

Dual reuptake inhibitors (“SNRIs”) are another class of compounds used as antidepressants. SNRIs increase the extracellular levels of both 5-HT and NE neurotransmitters in tissues of the nervous system receiving noradrenergic and/or serotonergic innervation by inhibiting the reuptake of both neurotransmitters, and increasing the levels of both neurotransmitters available to bind to the appropriate, respective postsynaptic transmitter.

Amphetamine belongs to a different class of drugs and it acts on the Central Nervous System (“CNS”) through two different pharmacological mechanisms. One mechanism consists in the inhibition of neuronal reuptake of NE and DA to prolong their concentration and time in the synaptic cleft. The second mechanism includes the ability to cause neuronal release of the three principle monoamine neurotransmitters DA, NE and 5-HT.

While an extensive number of anti-depressants have been approved for monotherapy in MDD, many patients remain symptomatic and disabled by incomplete resolution of MDD symptoms despite treatment with anti-depressant monotherapy at appropriate doses and trial duration. These patients with an inadequate therapeutic response represent up to two-thirds of patients treated with anti-depressant monotherapy. Commonly, these inadequate responders remain symptomatic with respect to mood, concentration, motivation, and interest, often reflected by disability in multiple life domains (e.g., work/school, social life, and home/family life). The present invention addresses this need.

### SUMMARY OF THE INVENTION

Accordingly, the present invention provides a method for treating a patient with depression or a depressive disorder, comprising administering an effective amount of an antidepressant in combination with a stimulant, or a derivative or analog thereof, to a patient in need thereof.

Accordingly, the present invention provides a method of treating depression or a depressive condition, e.g., major depression, in a patient, comprising: administering an antidepressant for a first period of at least about 4 weeks, 6 weeks, 8 weeks or 10 weeks, or any interval therebetween, and thereafter administering an effective amount of a stimulant in combination with said antidepressant to a patient in need of such treatment.

Accordingly, the present invention also provides a method of treating depression or a depressive condition in a patient in which there is administered an antidepressant for a first period of at least about 4 weeks and thereafter administering an effective amount of a stimulant in combination with the antidepressant to a patient in need of such treatment.

Accordingly, the present invention also provides a combination therapy of an antidepressant and a stimulant, or a derivative or analog thereof, for use in the treatment of depression or a depressive disorder.

Accordingly, the present invention also provides a combination therapy of an antidepressant and a stimulant, or a derivative or analog thereof, for use in the treatment of depression or a depressive disorder, e.g., major depression, wherein the combination therapy comprises (i) administration of an antidepressant for a first period of at least about 4 weeks, and (ii) thereafter administration of the combination of a stimulant and said antidepressant.

Accordingly, the present invention also provides a combination product comprising an antidepressant and a stimulant for use in the treatment of depression or a depressive disorder, e.g. major depression.

Accordingly, the present invention also provides a kit comprising: an antidepressant in a first dosage form, a stimulant in a second dosage form and a container for containing the first and second dosage forms. The kit may be used in the treatment of depression or a depressive disorder, e.g. major depression.

Accordingly, the present invention also provides a stimulant for use in the treatment of depression or a depressive disorder, e.g. major depression, wherein said stimulant is for administration in combination with an antidepressant.

Accordingly, the present invention also provides a stimulant for use in the treatment of depression or a depressive disorder, e.g. major depression, wherein said stimulant is for administration in combination with an antidepressant following a first period of administration of said antidepressant without said stimulant.

The invention comprises numerous embodiments and it is to be understood that the embodiments defined herein may be used independently or in conjunction with any definition, claim or any other embodiment defined herein.

In an embodiment, the depressive disorder is a major depressive disorder.

In an embodiment, the antidepressant is preferably a selective serotonin reuptake inhibitor (SSRI).

In an alternative embodiment, the antidepressant is a dual reuptake inhibitor (SNRI).

In an embodiment, when the antidepressant is an SSRI, the antidepressant is selected from the group consisting of: citalopram (Celexa®), dapoxetine (Priligy®) escitalopram (Lexapro®, Cipralex®), femoxetine (Malexil®), fluoxetine (Prozac®), fluvoxamine (Luvox), indalpine (Upstene®), paroxetine (Paxil®, Seroxat®), sertraline (Zoloft®, Lustral®) and zimelidine (Normud®, Zelmid®) and others that may be known to the art. Preferably, the SSRI is citalopram or escitalopram.

In an embodiment, when the antidepressant is an SNRI, the antidepressant is selected from the group consisting of: duloxetine (Cymbalta®), venlafaxine (Effexor®, Effexor XR®), desvenlafaxine (Pristiq®), milnacipran (Ixel®, Savella) and others that may be known to the art.

In a preferred embodiment the antidepressant is citalopram or escitalopram.

In an embodiment, the stimulant is selected from the group consisting of: amphetamine, phentermine, methamphetamine, methylphenidate, or mixtures thereof. In certain embodiments of the invention, the stimulant includes a covalently attached amino acid or amino acid containing compound. The stimulant may also include a covalently attached peptide that is optionally attached to the stimulant compound through the C-terminus of the peptide. The stimulant is preferably bound to lysine. More preferably, the stimulant is lisdexamfetamine.

Preferably, the antidepressant is citalopram or escitalopram and the stimulant is lisdexamfetamine.

In a further embodiment, the invention allows for the antidepressant to be administered simultaneously with the stimulant, e.g., in a single dosage form, although separate formulations and dosing schedules for the antidepressant and stimulant may also be employed, e.g., wherein the stimulant is administered before or after the antidepressant.

In a further embodiment, the combination therapy for use in the treatment of depression or a depressive disorder involves simultaneous administration of the antidepressant and the stimulant, e.g., in a single dosage form or two dosage forms administered at the same time. For example, the antidepressant may be escitalopram which may be co-administered (administered at the same time) with the stimulant.

In a further embodiment, the combination therapy for use in the treatment of depression or a depressive disorder involves sequential administration of the antidepressant and the stimulant, e.g., in two dosage forms administered one after the other.

In an alternative further embodiment, the combination therapy for use in the treatment of depression or a depressive disorder involves the separate administration of the antidepressant and the stimulant. For example, separate formulations and dosing schedules for the antidepressant and stimulant may also be employed, e.g., wherein the stimulant is administered before or after the antidepressant.

In an embodiment, the first period of the combination therapy is at least about 4 weeks, 5 weeks, 6 weeks, 7 weeks, 8 weeks, 9 weeks or 10 weeks.

In an embodiment, the first period of the combination therapy is 4 weeks, 5 weeks, 6 weeks, 7 weeks, 8 weeks, 9 weeks or 10 weeks.

The artisan will be able to readily determine the appropriate dosages of antidepressants based on the patient's clinical condition, the labeling recommendations for each such medication and based on the clinical response of the patient. In an embodiment, the amount of escitalopram administered to the patient in need thereof ranges from about 2 to about 40 mg/day, more preferably from about 10 to about 30 mg/day, and in particular embodiments, at a dose of about 20 mg/day.

In an embodiment, the amount of citalopram administered to a patient in need thereof ranges from about 10 mg/day to about 40 mg/day, more preferably from about 20 mg/day to about 30 mg/day and even more preferably 20 mg/day.

Dosages for other SSRIs and/or SNRIs are readily determined by the artisan, as noted above, based on potency relative to escitalopram or citalopram, the labeling recommendations for each such medication and/or on the clinical response of the patient.

The artisan will also be able to readily determine the appropriate dosages of stimulant(s) based on the patient's clinical condition, need for augmentation, the labeling recommendations for each such stimulant and/or based on the clinical response of the patient. Preferably, the amount of lisdexamfetamine administered to the patient in need thereof ranges from about 10 to about 250 mg/day, more preferably from about 20 to about 70 mg/day, and in particular embodiments, from about 20 to about 50 mg/day.

In an alternative embodiment, the methods of the invention provide for a method for treating an animal, comprising co-administering a therapeutically effective amount of escitalopram or a derivative or analog thereof or a pharmaceutically acceptable salt thereof and a therapeutically effective amount of stimulant or a derivative or analog thereof, or a pharmaceutically acceptable salt thereof. In certain embodiments, the animal is a human.

While amphetamine prodrugs are effective in augmenting the efficacy of SSRIs, we have found certain SSRIs to demonstrate an enhanced effect in combination with an amphetamine prodrug such as L-lysine-d-amphetamine. The prodrug is a conjugate in which amphetamine is covalently bound to an organic chemical species preferably such as an amino acid or a peptide containing from 1 to 10 amino acids. The amino acids are preferably independently selected at each occurrence from the naturally occurring amino acids.

### DETAILED DESCRIPTION OF THE INVENTION

As used herein the “anti-depressant effect” refers to the clinical assessment of improved symptoms or signs of depression.

As used herein, a “pharmaceutical composition” refers to any one or combination of two, three or more components, including the two active components which may be present in the same or different formulations. It may be in form of, for example, tablets, capsules, caplets, oral solutions and oral suspensions.

As used herein, a “mammal” and “animal” each preferably refers to humans although any mammal which could benefit from the combination therapy described herein is contemplated.

For all of the methods described herein, the identified compounds are contemplated to be employed in combination, simultaneously, or sequentially (e.g. in the same composition or in separate compositions).

It is thus to be understood that the term “combination” envisages the simultaneous, sequential or separate administration of the active components of the combination. Preferably the components are administered simultaneously. Conveniently, this normally occurs in one or more unit dosage forms containing both active components. Where the administration of those agents is sequential or separate, the delay in administering the second component should not be such as to lose the benefit of the synergistic or augmentation effect of the therapy.

The invention includes in a first aspect methods of treating a patient with or having depression or a depressive disorder, such as major depressive disorder. The methods include administering an effective amount of an antidepressant in combination with a stimulant or a derivative or analog thereof a patient in need thereof.

Accordingly, the present invention provides a combination therapy of an antidepressant and a stimulant, or a derivative or analog thereof, for use in the treatment of depression or a depressive disorder.

The antidepressant used in the invention is preferably a selective serotonin reuptake inhibitor (SSRI), such as citalopram or escitalopram and escitalopram is preferred.

As mentioned above, the invention includes administering a stimulant. One such class of stimulant useful in the invention is stimulants which include a covalently attached amino acid or amino acid containing compound such as a covalently attached peptide, which is preferably attached to the stimulant compound through the C-terminus of the peptide, and described in more detail below.

The invention includes treating depression or a depressive condition in a patient by using a staged treatment regimen which comprises: administering an antidepressant for a first period of at least about 4 weeks and thereafter administering an effective amount of a stimulant in combination with the antidepressant to a patient in need of such treatment. The first treatment period can be preferably at least about 8 weeks before the stimulant is administered. The stimulant is administered for at least about 6 weeks in combination with said antidepressant.

One preferred embodiment includes first administering the antidepressant citalopram or escitalopram at therapeutic doses for at least about 4, but more preferably 8 weeks and thereafter introducing the stimulant such as the preferred lisdexamfetamine as part of a combination therapy using the SSRI for at least about 6 weeks.

There is also provided a method for the treatment of an animal, e.g. a human, comprising co-administering a therapeutically effective amount of escitalopram or a derivative or analog thereof or a pharmaceutically acceptable salt thereof and a therapeutically effective amount of stimulant or a derivative or analog thereof, or a pharmaceutically acceptable salt thereof. In all aspects, the treatment combination can be continued for as long as chemical benefits are observed.

The SSRIs used in the invention are those well known to those of ordinary skill in the art and their therapeutic indications when administered alone are well documented. For brevity, the approved uses of the stated SSRIs are not listed here. Suitable SSRIs that may be used in the present invention include, for example, citalopram, escitalopram, dapoxetine, femoxetine, fluoxetine, fluvoxamine, ifoxetine, paroxetine, sertraline, zimelidine, etc, and mixtures thereof. Some preferred SSRIs are selected from among citalopram, escitalopram, dapoxetine, femoxetine, fluvoxamine, ifoxetine, paroxetine, sertraline and zimelidine. The SSRIs may be selected from among: citalopram, escitalopram, paroxetine and sertraline. In an alternative embodiment, preferred SSRIs include, for example, escitalopram or citalopram. In one embodiment, escitalopram is the preferred SSRI. The SSRIs are administered in amounts which are generally regarded as safe and effective for the treatment of depression, MDD or other depressive conditions for which the SSRI has been approved or is deemed to be desirable. The artisan is well aware of the dosing guidelines and the prescribing information available as part of the respective package inserts which provides the same is incorporated herein by reference.

The invention may be carried out using escitalopram, i.e., the pure (S) enantiomer of racemic citalopram ((S)-1-[3-(dimethylamino)propyl]-1-(4-fluorophenyl)-1,3-dihydroisobenzofuran-5-carbonitrile), or salts thereof. The amount administered in this embodiment is an effective amount of escitalopram, i.e. an amount which is non-toxic but sufficient to demonstrate a cognizable anti-depressant effect in mammals, with the range being generally from about 1 mg to about 50 mg a day, preferably from about 5 mg to about 30 mg a day, and more preferably from about 10 mg to about 20 mg a day. In one preferred embodiment, the effective amount of escitalopram is based on the amount of escitalopram oxalate (Lexapro®) ranging from about 10 mg to about 20 mg/day. In an alternative embodiment, an escitalopram salt is administered in an amount substantially equivalent to the amount of escitalopram of escitalopram oxalate.

In another embodiment, the effective amount of citalopram, based on citalopram hydrobromide, that is administered to a patient in need thereof ranges from about 10 mg/day to about 40 mg/day, more preferably from about 20 mg/day to about 30 mg/day and even more preferably is about 20 mg/day.

The amphetamine prodrugs included in the invention preferably include amphetamine covalently bound to a chemical moiety, such as those as described in U.S. Pat. Nos. 7,223,735 and 7,105,486, (the '735 and '486 patents), the contents of which are incorporated herein by reference and these amphetamine conjugates specifically form part of the disclosure of the present invention in terms of the amphetamine component of the combination. The '735 and '486 patents describe covalent attachment of amphetamine and derivatives or analogs thereof to a variety of chemical moieties. The chemical moieties may include any substance which results in a prodrug form, i.e., a molecule which is converted into its active form in the body by normal metabolic processes. The chemical moieties may be for instance, amino acids, peptides, glycopeptides, carbohydrates, nucleosides, or vitamins and the unattached portion of the carrier/conjugate may be in a free and unprotected state, or in the form of an ester or salt thereof.

In one embodiment, the amphetamine is attached to a single amino acid which is either naturally occurring or a synthetic amino acid. In a preferred embodiment, the or each amino acid is a naturally occurring amino acid. The conjugate may contain from 1 to 10 amino acids in one preferred embodiment. In an embodiment, the amphetamine is attached to a dipeptide or tripeptide, which could be any combination of the naturally occurring amino acids and synthetic amino acids. In another preferred embodiment of the invention, the amino acids are selected from L-amino acids for digestion by proteases. One particularly preferred L-amino acid prodrug of amphetamine useful in the methods described herein is the L-lysine-d-amphetamine or (N-[(1S)-1-methyl-2-phenylethyl]-L-lysinamide, or lisdexamfetamine dimesylate sold under the trademark Vyvanse® by Shire Pharmaceuticals.

The amount of amphetamine prodrug included is described as an effective amount, i.e. an amount which augments or enhances the effectiveness of the SSRI agent in the treatment of depression or depressive conditions, preferably MDD. The amount of amphetamine prodrug will vary somewhat, depending upon clinical conditions, but will be apparent to a clinician of ordinary skill without undue experimentation.

The dosing range of the L-lysine-d-amphetamine is normally in the range of 0.1 mg/kg to 75 mg/kg body weight per day in a single or divided doses. Preferably, the dosing range for L-lysine-d-amphetamine is in the range of from 0.1 mg/kg to 2 mg/kg of body weight. Similarly, the dosing range of the SSRI in the combination is in the range of 0.1 mg/kg to 75 mg/kg body weight per day, and preferably from 0.1 mg/kg to 1 mg/kg of body weight.

For purposes of illustration and not limitation, the amount of an amino acid prodrug of amphetamine which can be administered in accordance with the invention broadly ranges from about 5 mg to about 500 mg a day, and preferably from about 10 mg to about 250 mg a day. Preferably, the prodrug amphetamine is administered in a range from about 20 to about 70 mg/day, and more preferably, from about 20 to about 50 mg/day.

Thus, the amount of L-lysine-d-amphetamine administered according to the present invention preferably ranges from about 20 mg to about 70 mg a day. In one preferred embodiment, L-lysine-d-amphetamine is administered to patients in an amount of from about 20 mg to about 50 mg/day (e.g., 20 mg, 30 mg, 40 mg, 50 mg/day) based on the amount of L-lysine-d-amphetamine dimesylate.

Alternative stimulants can also include amphetamine salts, e.g., racemic amphetamine aspartate monohydrate, racemic amphetamine sulfate, dextroamphetamine saccharide, and dextroamphetamine sulfate and/or mixtures thereof. A mixture of amphetamine salts comprising the foregoing is available commercially as Adderall® and Adderall XR® from Shire Pharmaceuticals. Other alternative stimulants can include, e.g., methylphenidate or amphetamine, e.g., dextroamphetamine.

Conveniently, the dosages can be provided in unit dosage form containing both active components in the same form. The ratio of L-lysine-d-amphetamine to the SSRI (whether given in the same dosage form or separately) is in the range of 10:1 to 1:10 (weight:weight). More preferably, the ratio is in the range 5:1 to 1:2 and most preferably it is in the range 2:1 to 1:1 (weight:weight).

Both the SSRI and amphetamine prodrug will be administered using commonly available dosage forms. In many aspects, the SSRI and amphetamine prodrug will be administered in separate dosage forms to the mammal in need thereof. In other aspects, the two agents will be provided in a single dosage form which includes the combination. A non-limiting list of suitable dosage forms includes, for example, tablets, coated tablets, dragees, capsules, hard gelatin capsules, soft gelatin capsules, caplets, lozenges, oral solutions, oral suspensions or combinations thereof. The active ingredients may be mixed under sterile conditions with a pharmaceutically acceptable carrier and may be in aqueous or non-aqueous forms.

Preferred dosage forms are oral dosage forms such as tablets, capsules, caplets and lozenges. These improve patient compliance relative to other dosage forms.

The dosage forms may also contain any carriers or excipients such as diluents, binders and adhesives, lubricants, plasticizers, disintegrants, colorants, bulking substances, flavoring, sweeteners, buffers, adsorbents, etc. required for making a pharmaceutically acceptable dosage. For instance, the carriers or excipients may include microcrystalline cellulose, lactose, corn starch or derivatives thereof, talc, stearic acid or its salts etc. Suitable carrier materials for soft gelatine capsules can include, for example, vegetable oils, waxes, fats and semi-solid and liquid polyols. Suitable carrier materials for the production of solutions and syrups include, for example, water, polyols, sucrose, invert sugar and the like.

For the purposes of the present invention, augmentation is defined as the observation of a better effect with the combination of L-lysine-d-amphetamine together than is observed for either individual component at the same dose. The two active agents in the combination act in a different manner and hence the dosages are not additive. Consequently, an improvement relative to the individual dosages demonstrates synergy or augmentation.

### EXAMPLE 1

A clinical trial of Vyvanse® [(lisdexamfetamine dimesylate (or SPD489)-] as an adjunctive therapy in patients who have had an inadequate response to treatment with LEXAPRO (escitalopram) for Major Depressive Disorder (MDD) was carried out.

In this investigational 14-week, double-blind, randomized, placebo-controlled study (n=246), Vyvanse or placebo was administered orally as an adjunctive therapy to adults between the ages of 18 and 55, who experienced residual depressive symptoms following treatment with 20 mg/day escitalopram/LEXAPRO® (prospective 8-week period). Residual depressive symptoms were defined as 17—Item Hamilton Rating Scale for Depression (HAM-D 17) score at the end of this prospective phase (n=177). In the double blind 6-week period, those with residual depressive symptoms were randomized to receive escitalopram plus either placebo or Vyvanse (dose range of 20 to 50 mg per day) on a 1:1 basis.

The primary study endpoint was the mean change in the total MADRS score in non-remitters after 6 weeks of blinded treatment. Non-remitters were defined as having a total Montgomery-Asberg Depression Rating Scale (MADRS) score>10 at randomization, following 8 weeks of escitalopram treatment (n=129). The key secondary outcome was the Sheehan Disability Scale (SDS). Both the MADRS and SDS are well-validated and widely used assessments in MDD research; the MADRS provides evidence of symptomatic improvement while the SDS reflects the functional impact of the symptomatic improvement. The significance level was prospectively set at 10%.

Vyvanse demonstrated statistically significant improvement compared to placebo on the mean total MADRS of −2.2 [90% CI −4.4 to −0] after 6 weeks of treatment in the ‘per protocol’ analysis of non-remitting subjects (p=0.097). An additional pre-planned analysis was conducted among subjects who had a total MADRS score >10 and achieved <50% improvement in total MADRS score. This analysis found an endpoint LS mean difference between Vyvanse and placebo of −3.9 [90% CI −6.5 to −1.3; p=0.0132 (not adjusted for multiplicity)].

Analysis of the MADRS items revealed improvement present in domains of mood, concentration, interest, and motivation (all p≦0.08); no significant worsening on the MADRS items related to appetite, sleep, or thoughts of self-harm were present. The most commonly reported adverse events (dry mouth, headache, decreased appetite, and insomnia) in the trial were consistent with the known, labeled profile of VYVANSE in ADHD. Blood pressure and heart rate changes were also consistent with the product label. No notable ECG changes or changes in clinical laboratory assessments were reported in this study. The mean dose per day of VYVANSE used for adjunctive therapy with escitalopram was 29.6 mg.

The results of this example are significant because up to 60 percent of patients with MDD do not respond fully to standard anti-depressant pharmacotherapy, with up to two-thirds not achieving full remission. Thus, the combination of an SSRI with an amphetamine prodrug such as L-lysine-d-amphetamine, after an initial loading of the SSRI for several weeks provides improvements in the treatment of MDD.

### EXAMPLE 2

The procedure of Example 1 is repeated except that the adjunctive therapy with Vyvanse is carried out in adult patients who have an inadequate response to treatment after 8 weeks of therapy using citalopram/Lexapro at 40 mg/day for MDD. The patients are randomized to receive escitalopram plus either placebo or Vyvanse (dose range of 20 to 50 mg per day) on a 1:1 basis.

### EXAMPLE 3

The study described by Examples 1 and 2, supra was completed and additional data obtained that confirmed the efficacy of LDX as augmenting the effectiveness of escitalopram in the treatment of major depressive disorder (MDD) in patients classified as nonremitters according to the Montgomery-Asberg Depression Rating Scale (MADRS). Example 3 describes the study in greater detail, with additional statistical analysis, relative to Examples 1 and 2.

### Methods

### Study Design

This was a multicenter, randomized, double-blind, parallel-group, placebo-controlled study in participants with major depressive disorder (MDD) conducted at 15 sites in the United States from July 2009 to August 2010. The study had 4 phases: screening and washout (if necessary), 8 weeks of open-label escitalopram oxalate, 6 weeks of double-blind LDX treatment as augmentation therapy to escitalopram, and a safety follow-up.

The study was conducted in accordance with guidelines set forth by the Declaration of Helsinki and International Conference on Harmonisation Good Clinical Practice. After explanation of the study to participants, voluntary informed consent was obtained in writing.

### Study Participants

Eligible participants were adults (18-55 y) with a primary diagnosis of nonpsychotic MDD according to the Structured Clinical Interview for the Diagnostic and Statistical Manual of Mental Disorders, Fourth Edition—Text Revision™ disorders—Axis I (SCID-I) (American Psychiatric Association (Diagnostic and Statistical Manual of Mental Disorders, Fourth Edition Text Revision, Washington D.C., American Psychiatric Association, 2000). Diagnosis was confirmed using Module A of the Mini-International Psychiatric Interview (MINI). Participants not receiving antidepressants at screening were required to have a baseline Hamilton Rating Scale for Depression-17 items (HAM-D17) score 22; those receiving antidepressants at screening had to have a HAM-D17 score 0 and not to have achieved remission based on investigator opinion following at least 6 weeks of treatment. Women of childbearing age were required to be nonpregnant/nonlactating and on adequate contraception. Permitted concomitant therapies included hormonal therapy, thyroid medication, hypertensive monotherapy, bronchodilator inhalers, nonsedating antihistamines, antibiotics, and over the counter medications not affecting blood pressure (BP), heart rate, or the central nervous system.

Exclusion criteria included a current MDD episode that has not responded to adequate antidepressant treatment (wks of treatment within the typical maximum adult therapeutic range) or a lifetime history of antidepressant-resistant MDD; those with attention deficit hyperactivity disorder as assessed by historical diagnosis and a screening questionnaire; a severe comorbid psychiatric disorder (Axis I, Axis II, or other) assessed by SCID-I that in the investigators' opinion would contraindicate LDX treatment or confound efficacy/safety assessments; a first-degree relative with bipolar disorder, concurrent chronic or acute medical illness, disability, or other condition that might confound safety assessments or increase risk to the participant; a history of or current suicide risk, suicide attempts, or suicidal ideations; a history of seizures (exception, infantile febrile seizures); current diagnosis or history of Tourette disorder; current abnormal thyroid function or glaucoma; family history of sudden cardiac death or ventricular arrhythmias; history of symptomatic cardiovascular disease or structural cardiac abnormalities including cardiomyopathy, coronary artery disease, stroke, or moderate to severe hypertension (or resting systolic BP [SBP]>139 mmHg or diastolic BP [DBP]>89 mmHg); history (≦6 mo prior) of suspected substance abuse or dependence disorder; or known hypersensitivity to amphetamine, escitalopram oxalate, or citalopram. Prohibited concomitant therapies included investigational compounds, antidepressants other than escitalopram, antipsychotics, anxiolytics, antihistamines, clonidine, electroconvulsive therapy, guanfacine, herbal preparations, monoamine oxidase inhibitors, multiple antihypertensive agents, norepinephrine reuptake inhibitors, oral corticosteroids, psychostimulants, sedatives, and triptans.

### Treatment

After screening/washout, participants underwent 8 weeks of open-label escitalopram monotherapy (10 mg/d at week 1; 20 mg/d thereafter). At week 8 (the augmentation baseline), those with a tolerable safety profile and residual MDD symptoms, defined as a HAM-D17 score were randomized 1:1 to receive LDX or placebo in a 6-week double-blind phase. Participants were stratified by baseline remission status (escitalopram remitters: Montgomery-Asberg Depression Rating Scale [MADRS] total score 10; escitalopram nonremitters: MADRS score >10) to enhance between-treatment balance. Treatment with LDX was initiated at 20 mg/d. The dose was increased weekly, first to 30 mg/d and then to 50 mg/d, during the dose-optimization period. Investigators could increase the dose through week 10 and decrease the dose (only once per participant) at any time; the LDX dose at week 11 was maintained through study endpoint (week 14/final study visit). Participants returned 7 (±2) days after last dose of study medication for collection of adverse event (AE) and concomitant medication information.

### Efficacy Assessments and Endpoints

The primary efficacy measure was mean change in MADRS (Montgomery S A, Asberg M., Br J Psychiatry;134:382-389) total score from augmentation baseline to study endpoint in escitalopram nonremitters. On the MADRS, response levels were defined as 25% or 50% reductions in total score from augmentation baseline; remission was defined as a total score ≦10.

Further evaluation of MADRS outcomes in escitalopram nonremitters indicated that a significantly greater proportion of participants had a 50% decrease in MADRS total score with LDX versus placebo (66.2% vs 50.0%; P=0.0754) at study endpoint (Table 1). In addition, numerically greater proportions of escitalopram nonremitters achieved a 25% decrease in MADRS total score with LDX versus placebo (89.2% vs 81.3%; P=0.2236) and achieved MADRS remission (LDX, 49.2%; placebo, 34.4%; P=0.1088) at study endpoint.

For the full analysis set (FAS) defined as subjects who took at least one randomized study drug and had at least one MADRS assessment after the randomization, all MADRS outcomes indicated numerically greater improvement with LDX versus placebo but none reached the a priori threshold for statistical significance, as shown by Table 1, below.

TABLE 1
Effects of Randomized Treatment With LDX and Placebo on Depressive Symptoms Assessed by
MADRS, HAM-D17, and QIDS-SR in Escitalopram Nonremitters and All Randomized Participants

| | | | Escitalopram Nonremitters | | | All Randomized Participants | | |
|---|---|---|---|---|---|---|---|---|
| | Measure | Statistic | LDX (n = 65) | Placebo (n = 64) | P value | LDX (n = 88) | Placebo (n = 85) | P value |
| MADRS | Change in | LS mean | −7.1 (−8.7, −5.6) | −4.9 (−6.4, −3.3) | 0.0902 | −4.8 (−6.3, −3.4) | −3.5 (−4.9, −2.0) | 0.2101 |
| | total score | change | | | | | | |
| | | (90% Cl) | | | | | | |
| | 50% | n (%) | 43 (66.2) | 32 (50.0) | 0.0754 | 64 (72.7) | 52 (61.2) | 0.1038 |
| | decrease at | | | | | | | |
| | week 14 | | | | | | | |
| | MADRS | n (%) | 32 (49.2) | 22 (34.4) | 0.1088 | 52 (59.1) | 40 (47.1) | 0.1049 |
| | total score | | | | | | | |
| | ≦10 at week | | | | | | | |
| | 14 | | | | | | | |
| HAM-D17 | Change in | LS mean | −4.9 (−6.0, −3.9) | −4.0 (−5.1, −2.9) | 0.3091 | −3.9 (−5.0, −2.9) | −3.4 (−4.5, −2.4) | 0.5109 |
| | total score | change | | | | | | |
| | | (90% Cl) | | | | | | |
| | 50% | n (%) | 41 (63.1) | 35 (54.7) | 0.3736 | 62 (70.5) | 55 (64.7) | 0.4376 |
| | decrease at | | | | | | | |
| | week 14 | | | | | | | |
| | HAM-D17 | n (%) | 21 (32.3) | 17 (26.6) | 0.5633 | 37 (42.0) | 33 (38.8) | 0.7073 |
| | total score | | | | | | | |
| | ≦10 at week | | | | | | | |
| | 14 | | | | | | | |
| QIDS-SR* | Change in | LS mean | −2.4 (−3.1, −1.6) | −1.2 (−2.0, −0.4) | 0.0774 | −2.5 (−3.1, −1.8) | −1.2 (−1.9, −0.6) | 0.0203 |
| | total score | change | | | | | | |
| | | (90% Cl) | | | | | | |

*For QIDS-SR, n = 63 and n = 62 for escitalopram nonremitters in the LDX and placebo groups, and n = 85 and n = 83 for all randomized LDX and placebo participants, respectively.
HAM-D17 (Table 1)
For escitalopram nonremitters, augmentation with LDX and placebo decreased HAM-D17 total scores from weeks 9-14 compared with week 8.
Change in HAM-D17 scores from week 8 to 14 were numerically greater with LDX vs placebo; these changes were not statistically significant (Table 1)
QIDS-SR
For escitalopram nonremitters, augmentation with LDX significantly decreased QIDS-SR total scores vs placebo at week 14 compared with week 8 (P = .0774) (Table 1)
For escitalopram nonremitters, LS mean (90% CI) change in QIDS-SR total scores at week 14 were −1.9 (−2.9, −0.9) and −0.4 (−1.4, −0.5) for those taking LDX and placebo, respectively (P = .0852)
CGI
At week 14, LS mean (SE) CGI-I scores were similar in both groups (LDX, 2.4 [0.14]; placebo, 2.6 [0.14]; P = .3199)
However, CGI-S scores at week 14 showed a numerically greater proporation of patients had a response ranging from no illness, to mild illness, with LDX (88.9%) vs placebo (61.3%)

### Safety Assessments

Adverse events (AEs) were collected during the study and coded using the Medical Dictionary for Regulatory Activities, Version 11.1. Treatment-emergent AEs (TEAEs) were defined as AEs that started or deteriorated on or after the date of the first dose of study drug during the double-blind treatment phase and no later than 3 days following the last dose of study drug. The incidence of TEAEs of special interest, including psychiatric (psychosis/mania, suicide, aggression, or other) and nonpsychiatric (weight, clinical laboratory [liver enzymes], vital signs, and sexual dysfunction) was also assessed. Vital signs (sitting SBP and DBP, pulse, and sitting respiratory rate), and Columbia-Suicide Severity Rating Scale (C-SSRS; Posner K, Brent D, Lucas C, et al.) a semistructured interview assessing the occurrence, severity, and frequency of suicide-related thoughts and behaviors) were measured at screening and all study visits. Laboratory assessments were conducted at screening, week 8, week 11, and study endpoint; physical examinations were conducted at screening, week 8, and study endpoint.

### Statistical Analysis

The primary efficacy analysis was performed on the primary efficacy analysis set, which was defined as all escitalopram nonremitters who had ≧1 MADRS assessment after starting randomized LDX augmentation and who took ≧1 dose of LDX or placebo.

The primary efficacy assessment was analyzed using an analysis of covariance (ANCOVA) model, with randomized augmentation treatment group as a factor and MADRS total score at augmentation baseline as a covariate. Similarly, ANCOVA models were used to assess changes in HAM-D17, QIDS-SR, and CGI-I scores. Primary efficacy analyses were performed at the pre-specified 2-sided significance level of 0.10 for this exploratory study. The percentage of participants achieving 25% or 50% response, based on MADRS and HAM-D17 total score, were summarized by visit during double-blind treatment and compared between randomized augmentation treatment groups using the Cochran-Mantel-Haenszel test; these data were stratified by augmentation baseline remission status.

Safety analyses during double-blind treatment were performed on the randomized safety analysis set (all participants who took dose of randomized augmentation treatment and had safety assessment after treatment initiation). AEs, vital signs, weight and body mass index (BMI), electrocardiograms (ECG) findings, laboratory results, and C-SSRS results were descriptively summarized.

### Results

### Disposition, Demographics, and Baseline Characteristics

A total of 239 of 246 enrolled participants received open-label escitalopram and were included in the full safety analysis set. Mean (SD) MADRS and HAM-D17 total scores were 32.6 (4.90) and 25.1 (3.07), respectively, at open-label baseline and 16.9 (8.59) and 13.7 (6.07), respectively, at augmentation baseline for the randomized safety set (n=173). Mean (SD) change from open-label baseline in MADRS total score with escitalopram (n=239) at augmentation baseline (week 8) was −15.6 (9.66).

### Extent of Exposure

The mean (SD) daily LDX dose during double-blind treatment was 29.6 (9.69) mg. During double-blind treatment, all 88 participants received the 20-mg dose; 75% (66/88) had their dose increased to 30 mg, and 42% (37/88) had their dose increased to 50 mg. The mean (SD) duration of exposure was 38.3 (9.56) days for LDX and 40.0 (6.60) days for placebo. Approximately 70% of participants in each group were treated for 36-42 days.

### Primary Efficacy Endpoint

### Montgomery-Asberg Depression Rating Scale

The primary efficacy analysis showed that among escitalopram nonremitters, LDX augmentation produced a significantly greater reduction in mean MADRS total score from augmentation baseline to study endpoint versus placebo (least squares mean [90% CI]: −7.1 [−8.7, −5.6] vs −4.9 [−6.4, −3.3]; P=0.0902).

### Escitalopram Remitters

Among escitalopram remitters, at augmentation baseline and study endpoint there were no significant differences in primary or secondary efficacy outcome measures between LDX and placebo treatment groups.

### Safety

### Adverse Events

Safety data for open-label treatment with escitalopram have been reported elsewhere (Geibel B, et al. Demographics and open-label escitalopram therapy in adults with major depressive disorder: prior to adjunctive lisdexamfetamine dimesylate or placebo. Presented at: 164th Annual Meeting of the American Psychiatric Association; May 14-18, 2011; Honolulu, HI). During randomized augmentation phase, 53 (60.2%) participants receiving LDX reported TEAEs, including 16 (18.2%), 27 (30.7%), and 10 (11.4%) participants reporting mild, moderate, and severe TEAEs, respectively. A total of 42 (49.4%) participants receiving placebo experienced TEAEs, including 16 (18.8%), 22 (25.9%), and 4 (4.7%) participants reporting mild, moderate, and severe TEAEs, respectively. A serious TEAE, severe rhabdomyolysis that resolved after 20 days, was reported in a placebo participant. Study discontinuation due to TEAEs occurred in 3 participants (LDX, n=2; placebo, n=1). No deaths were reported during the study. TEAEs occurring in 5% of participants during double-blind treatment are shown in Table 2, below.

TABLE 2
TEAEs Reported During the Double-Blind Augmentation
Phase, Randomized Safety Analysis Set

| | LDX | Placebo |
|---|---|---|
| Adverse Event, n (%) | (n = 88) | (n = 85) |
| Any TEAE | 53 (60.2) | 42 (49.4) |
| Any TEAE occurring at a frequency of >5% | | |
| Dry mouth | 10 (11.4) | 0 |
| Headache | 10 (11.4) | 4 (4.7) |
| Decreased appetite | 6 (6.8) | 2 (2.4) |
| Nasopharyngitis | 5 (5.7) | 3 (3.5) |
| Insomnia | 4 (4.5) | 6 (7.1) |

LDX = lisdexamfetamine;
TEAEs = treatment-emergent adverse events.

Psychiatric TEAEs of special interest were experienced by 16 (18.2%) and 14 (16.5%) participants with LDX and placebo, respectively. Psychiatric TEAEs occurring in participants in either group (LDX vs placebo) included feeling jittery (2 [2.3%] vs 1 [1.2%]), irritability (3 [3.4%] vs 2 [2.4%]), somnolence (0 vs 3 [3.5%]), abnormal dreams (1 [1.1%] vs 2 [2.4%]), anxiety (3 [3.4%] vs 1 [1.2%]), and insomnia (4 [4.5%] vs 6 [7.1%]); no participant had a psychiatric TEAE categorized as psychosis/mania, suicidal, or aggression. Nonpsychiatric TEAEs of special interest were experienced by 8 (9.1%) participants receiving LDX and 6 (7.1%) receiving placebo. For participants receiving LDX, 2 (2.3%) experienced decreased weight, 1 (1.1%) had abnormal hepatic enzymes, and 5 (5.7%) had vital-sign related TEAEs. For participants receiving placebo, 1 (1.2%) experienced increased weight, 5 (5.9%) had liver enzyme abnormalities, and 1 (1.2%) had decreased libido.

### Columbia-Suicide Severity Rating Scale

At screening and open-label baseline, 43 (18.0%) and 18 (7.5%) participants answered “yes” to the question: “Have you wished you were dead or wished you could go to sleep and not wake up?” During double-blind treatment, an affirmative response was provided by 1 participant receiving LDX at week 9, 2 at week 10, and 1 at week 14 and by 1 placebo participant each at weeks 9, 10, 12, and 14. No participants exhibited suicidal behavior during augmentation treatment.

### Vital Signs and Electrocardiogram Findings

Small mean changes from augmentation baseline were seen for BP, pulse rate, and weight at study endpoint (Table 3). ECG findings indicated that heart rate tended to increase more and Fridericia-adjusted QT interval tended to decrease more with LDX than with placebo, as illustrated by Table 3, below.

TABLE 3
Mean (SD) Change From Augmentation Baseline in Vital Signs
and ECG at Visit 13 (Week 14), Randomized Safety Analysis Set

| | | LDX | Placebo |
|---|---|---|---|
| Vital signs | SBP, mmHg | 2.3 (9.04) | 0.5 (8.98) |
| | DBP, mmHg | 0.9 (6.61) | −1.0 (7.19) |
| | Pulse, bpm | 3.3 (8.45) | −0.4 (7.10) |
| | Weight, kg | −1.2 (2.00) | 0.3 (2.05) |
| | BMI, kg/m² | −0.4 (0.70) | 0.1 (0.70) |
| ECG findings | HR, bpm | 4.8 (8.64) | −0.4 (7.39) |
| | QTcF, ms | −4.9 (11.84) | −1.6 (11.23) |

BMI = body mass index;
ECG = electrocardiogram;
DBP = diastolic blood pressure;
HR = heart rate;
LDX = lisdexamfetamine;
SBP = systolic blood pressure;
QTcF = Fridericia-adjusted QT interval.

BMI remained stable during double-blind treatment. For participants on LDX, 81.8%, 76.9%, and 82.5% of patients categorized as normal (18.5 to <25.0), overweight (25.0 to <30.0), or obese (30), respectively, at augmentation baseline remained so at study endpoint; 91.7%, 89.7%, and 93.5% of participants on placebo in the normal, overweight, and obese BMI ranges, respectively, at augmentation baseline remained so at study endpoint. While these data might suggest that some participants shifted BMI categories, apparent shifts may be largely due to missing endpoint values; shifts that were observed were mostly to lower BMI categories.

No changes in mean clinical laboratory values over time were seen that were of clinical concern. No significant adverse effects were seen that went beyond those previously seen with escitalopram and LDX.

Main Outcome Measures: The primary endpoint was change in MADRS total score in escitalopram nonremitters (MADRS total score >10) from augmentation baseline to study endpoint using last observation carried forward. Other measures included changes in the Hamilton Rating Scale for Depression-17, Clinical Global Impression (CGI)-Severity and CGI-Improvement scales, and Quick Inventory of Depressive Symptomology-Self Report and assessment of clinical laboratory values, physical examinations, the Columbia-Suicide Severity Rating Scale, and adverse events (AEs).

Results: In escitalopram nonremitters, the least square mean (90% CI) reduction in MADRS total score from augmentation baseline to study endpoint was significantly greater with LDX vs placebo (−7.1 [−8.7, −5.6] vs −4.9 [−6.4, −3.3]; P=0.0902); no treatment differences were found for escitalopram remitters. Among participants receiving randomized treatment, 60.2% (53/88) on LDX and 49.4% (42/85) on placebo had at least 1 treatment-emergent AE (TEAE); 1 serious TEAE during randomized treatment occurred in a participant receiving placebo. TEAEs with an incidence 5% (LDX vs placebo) were dry mouth (11.4% vs 0%), headache (11.4% vs 4.7%), decreased appetite (6.8% vs 2.4%), nasopharyngitis (5.7% vs 3.5%), and insomnia (4.5% vs 7.1%). Mean (SD) changes from augmentation baseline to study endpoint in systolic and diastolic blood pressure and pulse (LDX vs placebo) were 2.3 (9.04) vs 0.5 (8.98) mmHg, 0.9 (6.61) vs −1.0 (7.19) mmHg, and 3.3 (8.45) vs −0.4 (7.10) bpm, respectively. No clinically significant mean changes were seen in the electrocardiogram and laboratory findings.

44 of 173 randomized participants with residual symptoms, based on HAM-D17 scores, were considered escitalopram remitters based on MADRS total scores, suggesting that many patients may continue to experience residual symptoms despite achieving overall remission criteria with treatment.

Adjusted effect size of MADRS total scores for LDX vs placebo at endpoint (−0.3) was similar to the effect size(0.228) predicted to achieve significant differences in the STAR*D analysis 10 and similar to effect size (0.35) for MADRS total scores for aripiprazole vs placebo as augmentation of standard antidepressant treatment11

LDX improved MDD symptoms vs placebo as determined by both clinician-assessed and patient self-reported measures for escitalopram nonremitters

LDX improved self-assessed MDD symptoms for escitalopram remitters

Simultaneous improvement in measures of both depressive symptoms and functional disability in non-remitting subjects suggests the treatment effect is clinically meaningful, in addition to being statistically significant.

No evidence of new or unique safety findings were observed with LDX in this population. Increases in blood pressure and pulse rate generally were small and consistent with product labeling. No significant adverse effects were seen that went beyond those previously seen with escitalopram and LDX.

Various references are cited throughout this application, the contents of which are incorporated by reference herein in their entireties.

1-27. (canceled)

28. A method of treating a patient having a major depressive disorder, comprising administering an antidepressant in combination with a stimulant or a derivative or analog thereof to the patient.

29. The method of claim 28, wherein the antidepressant is a selective serotonin reuptake inhibitor (SSRI).

30. The method of claim 29, wherein the selective serotonin reuptake inhibitor is citalopram or escitalopram.

31. The method of claim 30, wherein the selective serotonin reuptake inhibitor is escitalopram.

32. The method of claim 28, wherein the stimulant comprises a covalently attached amino acid or amino acid containing compound.

33. The method of claim 28, wherein the stimulant comprises a covalently attached peptide.

34. The method of claim 33, wherein the peptide is attached to the stimulant compound through a C-terminus of the peptide.

35. The method of claim 32, wherein the stimulant is amphetamine covalently bound to lysine.

36. The method of claim 35, wherein the stimulant is lisdexamfetamine.

37. The method of claim 28, wherein the stimulant is selected from the group consisting of racemic amphetamine aspartate monohydrate, racemic amphetamine sulfate, dextroamphetamine saccharide, and dextroamphetamine sulfate and mixtures thereof

38. The method of claim 28, wherein the antidepressant and the stimulant are administered in a single dosage form.

39. A method of treating a patient having a major depressive disorder, comprising:
administering an antidepressant to the patient for a first period of at least about 4 weeks and thereafter administering a stimulant in combination with the antidepressant to the patient.

40. The method of claim 39, wherein the antidepressant is administered for at least about 8 weeks before the effective amount of the stimulant is administered.

41. The method of claim 39, wherein the stimulant is administered for at least about 6 weeks in combination with said antidepressant.

42. The method of claim 39, wherein the antidepressant is citalopram or escitalopram and the stimulant is lisdexamfetamine.

43. The method of claim 42, wherein the escitalopram is administered to the patient in an amount of from about 1 to about 50 mg/day.

44. The method of claim 42, wherein the escitalopram is administered to the patient in an amount of from about 5 to about 30 mg/day.

45. The method of claim 42, wherein the scitalopram is administered to the patient in an amount of from about 10 to about 20 mg/day.

46. The method of claim 45, wherein escitalopram is administered to the patient in an amount of about 20 mg/day.

47. The method of claim 42, wherein the lisdexamfetamine is administered to the patient in an amount of from about 10 to about 250 mg/day.

48. The method of claim 42, wherein the lisdexamfetamine is administered to the patient in an amount of from about 20 to about 70 mg/day.

49. The method of claim 42, wherein the lisdexamfetamine is administered to the patient in an amount of from about 20 to about 50 mg/day.

50. The method of claim 42, wherein the citalopram is administered to the patient in an amount of from about 10 to about 40 mg/day.

51. A kit for practicing the method of claim 28 comprising: an antidepressant in a first dosage form, a stimulant in a second dosage form and a container for containing the first and second dosage forms.

52. A method treating an animal, comprising co-administering a therapeutically effective amount of escitalopram or a derivative or analog thereof or a pharmaceutically acceptable salt thereof and a therapeutically effective amount of stimulant or a derivative or analog thereof, or a pharmaceutically acceptable salt thereof.

53. The method of claim 52, wherein the animal is a human.
